(19) United States
(12) Patent Application Publication
(10) Pub. No.: US 2024/0252813 A1
(43) Pub. Date: Aug. 1, 2024

(54) SYSTEMS AND METHODS FOR TRANS-ESOPHAGEAL SYMPATHETIC GANGLION RECRUITMENT

(71) Applicant: Lungpacer Medical Inc., Vancouver (CA)

(72) Inventors: Thiago Gasperini BASSI, Burlington (CA); Joaquin Andres HOFFER, Anmore (CA); Steven Campbell REYNOLDS, North Vancouver (CA)

(73) Assignee: Lungpacer Medical Inc., Vancouver (CA)

(21) Appl. No.: 18/605,386

(22) Filed: Mar. 14, 2024

Related U.S. Application Data
(63) Continuation of application No. 17/165,231, filed on Feb. 2, 2021, now Pat. No. 11,944,810.
Application No. 17/165,231 is a continuation of application No. 16/054,273, filed on Aug. 3, 2018, now Pat. No. 10,940,308.
(60) Provisional application No. 62/541,652, filed on Aug. 4, 2017.

(51) Int. Cl. A61N 1/05; A61B 3/11; A61B 5/00; A61B 5/01; A61B 5/026; A61B 5/03; A61B 5/24; A61B 5/285; A61B 5/369; A61B 8/08; A61B 8/12; A61B 18/00; A61B 18/14

(52) U.S. Cl. CPC A61N 1/0517; A61B 5/037; A61B 5/24; A61B 5/285; A61B 8/0808; A61B 8/0816; A61B 18/1492; A61B 3/11; A61B 5/01; A61B 5/026; A61B 5/369; A61B 5/4233; A61B 8/12; A61B 2018/00488

(57) ABSTRACT

A method may include positioning a catheter, including at least one electrode, within an esophagus such that the electrode is proximate to at least one sympathetic ganglion. The methods may further include recruiting the sympathetic ganglion via an electrical signal, monitoring the recruitment of the sympathetic ganglion, and, based on the monitoring the recruitment of the sympathetic ganglion, adjusting the electrical signal from the at least one electrode.

### CROSS-REFERENCE TO RELATED APPLICATION(S)

This application claims priority to U.S. Provisional Application No. 62/541,652, filed on Aug. 4, 2017, the entire disclosure of which is hereby incorporated by reference in its entirety.

In general, all publications, patent applications, and patents mentioned in this specification are herein incorporated by reference in their entirety to the same extent as if each individual document was specifically individually indicated to be incorporated by reference.

### TECHNICAL FIELD

The embodiments of this disclosure generally relate to methods, systems, and devices for the diagnosis, mitigation, and treatment of neural injury (e.g., aneurism, stroke, concussion, etc.). In some examples, the present disclosure is directed to a method of reducing the occurrence of brain cell damage or death of a patient. One exemplary aspect is directed to a method of reducing the occurrence of brain cell damage or death caused by a transient cerebral hypoxia/ischemia condition, a brain inflammation condition, or a traumatic brain injury (TBI) event. Another exemplary aspect is directed to devices, systems, and methods for reducing brain and/or cognitive injury in patients diagnosed with a subarachnoid hemorrhage (SAH).

### BACKGROUND

SAH is characterized by bleeding into the space between the arachnoid membrane and the pia mater of the brain. SAH may occur as a result of a head injury or as a consequence of a cerebral aneurysm. The worldwide incidence of SAH has been estimated to be 7-25 per 100,000 person-years; from 1% to 7% of cerebrovascular accidents (CVAs, e.g., strokes) are the result of SAH. In some instances, SAH may result in delayed cerebral ischemia associated with a cerebral vasospasm (CVS). Studies have estimated 33% to 46% of patients with SAH will experience a clinically relevant CVS. Further, the delayed cerebral ischemia resulting from an SAH-related CVS may significantly impair a patient's functionality and quality of life, with only 20% of patients leaving the hospital with their pre-CVA neurological status.

Without being limited by theory, it is believed the combination of intracerebral hemorrhage and raised intracranial pressure may lead to an over-activation of the sympathetic system leading to an increased risk of CVS. This over-activation is thought to occur through two mechanisms, a direct effect on the medulla that leads to activation of the descending sympathetic nervous system and a local release of inflammatory mediators that circulate to the peripheral circulation where they activate the sympathetic system. The over-activation of the sympathetic system may result in a release of adrenaline, a sudden increase in blood pressure, increased contractility of the ventricle system, increased vasoconstriction, and/or increased systemic vascular resistance. Over-activation of the sympathetic system can result in cardiac arrhythmias, electrocardiogramanges, and/or cardiac arrest that may occur rapidly after the onset of hemorrhage. In some cases, a neurogenic pulmonary edema may develop which is characterized by increased pressure within the pulmonary circulation, leading to leaking of fluid from the pulmonary capillaries into the air spaces of the lung.

To limit the negative consequences resulting from SAH, physicians have experimented with sympathetic ganglion blocks in an attempt to prevent an over-activation of the sympathetic system. For example, an anesthetic may be delivered to the sympathetic ganglia via hypodermic needle using ultrasound visualization, fluoroscopy, or computed tomography (CT) scanning. In one procedure, a needle is inserted between the trachea and carotid sheath. In another procedure, a needle is inserted rostral to the sternoclavicular junction. Such invasive techniques involving the insertion of a needle into the neck and/or near the trachea are associated with the development of pneumothorax. Further, these techniques require large, immobile, and/or expensive imaging equipment and procedures, such as, for example, ultrasound visualization, fluoroscopy, or CT scanning.

Such procedures require specialized training, large-scale equipment and/or imagery machinery, and may only be performed in few hospitals with specialized staff. Further, such approaches are limited in the regions of cervical sympathetic ganglia that may be blocked due to the invasive nature of such procedures, the proximity of nerves, vasculature, and trachea, and the imprecision of anesthetic blocks. Additionally, the feasibility of using such techniques as a preventive measure is uncertain. The local anesthetics used in such blocks may last only several hours, while the increased risk of a delayed CVS may last up to 14 days after the SAH. Further, the long-term consequences of continued use of such anesthetics as nerve blocking agents are not yet known and are potentially severe.

### SUMMARY

Embodiments of the present disclosure relate to, among other things, systems, devices, and methods for treating, including preventing and moderating, brain injury. Each of the embodiments disclosed herein may include one or more of the features described in connection with any of the other disclosed embodiments.

This disclosure includes methods of treatment. In some aspects, the methods may include positioning a catheter within the esophagus, the catheter including at least one electrode, wherein the catheter is positioned so that the at least one electrode is proximate to at least one sympathetic ganglion. The methods may further include recruiting the sympathetic ganglion via an electrical signal from the at least one electrode. This may include recruiting at least one of a left stellate ganglion or a right stellate ganglion via the electrical signal from the at least one electrode. The recruiting of the sympathetic ganglion may include blocking the transmission of nerve signals along the sympathetic ganglion.

In some examples, the methods may further include where at least one electrode includes a plurality of electrodes. A first electrode of the plurality of electrodes may be positioned on a surface opposite a second electrode of the plurality of electrodes, where the first electrode and second electrode are positioned at the same axial level. In some aspects, an electrical signal may be transmitted by the plurality of electrodes to recruit the left stellate ganglion and the right stellate ganglion. In some examples, at least one electrode is positioned inferior to the C4 vertebra and superior to the T2 vertebra. In some aspects, a method may include positioning a catheter including at least four electrodes. The catheter may be positioned through an oroesophageal or a nasoesophageal cavity.

In some examples of methods, the electrical signal is pulsed and has a frequency of 100 Hz to 100 kHz and an amplitude of 10 μA to 20 mA. The methods may further include monitoring the recruitment of the sympathetic ganglia. The monitoring may include observing palpebral droop, performing an electroencephalogram, measuring pupil diameter, observing a color change in a sclera, measuring a skin temperature, measuring a skin perspiration, performing a transcranial Doppler ultrasonogram, or a combination thereof. In some aspects, based on the monitoring the recruitment of the sympathetic ganglia, the methods may further comprise adjusting the electrical signal from at least one electrode.

In further aspects, systems for treatment may include an esophageal catheter, one or more sensors for sensing a physiologic parameter indicative of a state of the sympathetic ganglia, and a controller in communication with the at least one electrode and the one or more sensors, where, upon receiving a first signal from the one or more sensors based on the sensed physiological parameter, the controller induces the at least one electrode to transmit an electrical signal that recruit the sympathetic ganglia. In some examples, the esophageal catheter comprises at least one electrode configured to be positioned within the esophagus proximate to at least one sympathetic ganglion. The one or more sensors may measure or monitor pupil dilation, skin temperature, skin perspiration, blood flow, electroencephalography, or a combination thereof. In other aspects, upon receiving a second signal from the one or more sensors, the controller induces the at least one electrode to cease transmitting the electrical signal or the controller induces the at least one electrode to adjust one or more of the amplitude or frequency of the transmitted electrical signal.

In some examples, the at least one electrode of the systems include a plurality of electrodes configured to independently transmit electrical signals and a first electrode of the plurality of electrodes is configured to be positioned proximate to a left stellate ganglion and a second electrode of the plurality of electrodes is configured to be positioned proximate to a right stellate ganglion. In some aspects, upon receiving a second signal from one or more sensors, the controller may induce one of the plurality of electrodes to adjust one or more of the amplitude or frequency of the electrical signal.

In further examples, an esophageal catheter may include a intermediate section operable to radially expand from a contracted state to an expanded state, a plurality of electrodes, and/or a feeding tube. At least two of the plurality of electrodes are farther displaced when the intermediate section is in an expanded state. The intermediate section may include an inflatable member. In some embodiments, the intermediate section may comprise at least two arms. The at least two arms may be connected by a tether and each arm may include a joined end, a proximal end, and at least one electrode positioned between the joined and the proximal end. The joined end may connect to the joined end of one or more other arms and the proximal end may be farther displaced from the proximal end of one or more other arms when the intermediate section is in an expanded state as compared to when the intermediate section is in a contracted state.

### BRIEF DESCRIPTION OF DRAWINGS

The accompanying drawings, which are incorporated in and constitute a part of this specification, illustrate non-limiting embodiments of the present disclosure and together with the description serve to explain the principles of the disclosure.

FIG. 1A illustrates the anatomy of selected tissues and anatomical lumens in a person's head and neck, along with an exemplary esophageal catheter placed within the esophagus, according to one or more embodiments of the present disclosure;

FIG. 1B illustrates the anatomy of selected tissues, anatomical lumens, and organs in a person's head, neck, and torso, along with an exemplary esophageal catheter placed within the esophagus, according to one or more embodiments of the present disclosure;

FIG. 2A illustrates an axial cross section including the anatomy of selected nerves, lumens, and bones in a person's neck, according to one or more embodiments of the present disclosure;

FIG. 2B illustrates the axial cross section shown in FIG. 2A, along with exemplary esophageal catheter electrodes placed within the esophagus;

FIG. 3A illustrates a perspective view of an esophageal catheter, according to one or more embodiments of the present disclosure;

FIG. 3B illustrates a cross-sectional view depicting the esophageal catheter shown in FIG. 3A, along line 3B-3B of FIG. 3A;

FIG. 4A illustrates a perspective view of an esophageal catheter in a contracted state, according to one or more embodiments of the present disclosure;

FIG. 4B illustrates a cross-sectional view depicting the esophageal catheter in a contracted state shown in FIG. 4A, along line 4B-4B of FIG. 4A;

FIG. 4C illustrates a perspective view of the esophageal catheter of FIG. 4A in an expanded state;

FIG. 4D illustrates a cross-sectional view depicting the esophageal catheter in an expanded state, along line 4D-4D of FIG. 4C;

FIG. 5A illustrates a perspective view of an esophageal catheter in a contracted state, according to one or more embodiments of the present disclosure;

FIG. 5B illustrates a perspective view of the esophageal catheter shown in FIG. 5A, in an expanded state;

FIG. 5C illustrates a cross-sectional view depicting the esophageal catheter in an expanded state shown in FIG. 5B, along line 5C-5C of FIG. 5B;

FIG. 6 illustrates a schematic view of a system having an esophageal catheter, a control unit, and one or more sensors, according various embodiments of the present disclosure; and

FIG. 7 is a flowchart of a method according to an embodiment of the present disclosure.

### DETAILED DESCRIPTION

Throughout the following description, specific details are set forth to provide a more thorough understanding to persons skilled in the art. The following description of examples of the technology is not intended to be exhaustive or to limit the system to the precise forms of any example embodiment. Accordingly, the description and drawings are to be regarded in an illustrative sense, rather than a restrictive sense.

Further aspects of the disclosures and features of example embodiments are illustrated in the appended drawings and/or described in the text of this specification and/or described in the accompanying claims. It may be understood that both the foregoing general description and the following detailed description are exemplary and explanatory only and are not restrictive of the invention, as claimed. As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” and “approximately,” indicate a range of values within +/−15% of a stated value.

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device or insertion device. When used herein, “proximal” refers to a position relatively closer to the exterior of the body or closer to an operator using the medical device or insertion device. In contrast, “distal” refers to a position relatively further away from the operator using the medical device or insertion device, or closer to the interior of the body.

One or more embodiments may relate to a device and/or method to deliver transesophageal neuromodulation to a cervical or thoracic sympathetic ganglion (e.g., a stellate ganglion) in order to increase brain perfusion. Given the proximity of the esophagus to target cervical sympathetic ganglia, an esophageal catheter including electrodes may be deployed within the esophagus to delivery high-frequency (e.g., greater than or equal to 1 kHz) electrical signals (e.g., electrical pulse trains).

In general, embodiments of this disclosure relate to systems, medical devices, and methods for recruiting sympathetic ganglia and preventing, modulating, controlling, or treating injury (e.g., injury of the brain). For example, the injury may be caused or enhanced by a TBI event. As used herein, the term “injury” may refer to an alteration in cellular or molecular integrity, activity, level, robustness, state, or other alteration that is traceable to an event. For example, brain injury may be neuronal injury resulting from stress (repetitive stress), inflammation, oxidative stress, disease, pain, stroke, and/or physical injury such as surgery or trauma (e.g., a TBI event). An SAH or a resulting CVS may exacerbate, worsen, or cause a brain injury. One or more methods herein may relate to treating and monitoring patients with an SAH to prevent or reduce incidence of a delayed CVS. Further, one or more methods herein may be applied to improve outcomes in patients with an acute stroke or a traumatic brain injury. Recruitment of at least one sympathetic ganglion may control, reduce or mitigate inflammation from ischemia or traumatic brain injury that may cause secondary injury to neurons.

In one or more embodiments, recruiting a nerve may include blocking the transmission of nerve signals along one or more cells (e.g. neurons). Recruiting a nerve may include transmitting an electrical signal (e.g., a recruitment signal) to the nerve that interferes with the transmission of a normal nerve signal. In some aspects, recruiting a nerve may be an ongoing process via the transmission of a repeating, pulsed, electrical signal to the nerve. The repeating, pulsed, electrical signal may stop axonal conduction of nerve signals. In instances, a nerve receiving an ongoing recruitment signal may be considered recruited. In some embodiments, a nerve which is unable to conduct nerve signals may be considered recruited. Ceasing the recruitment of a nerve may be referred to herein as releasing the nerve. Similarly, a nerve that is no longer recruited, but was once recruited, may be referred to as released.

In one or more embodiments, a method of treatment comprises positioning a catheter including at least one electrode within an esophagus such that the at least one electrode is proximate to at least one sympathetic ganglion, and recruiting the at least one sympathetic ganglion via an electrical signal from the at least one electrode. At least one electrode may be positioned inferior to the C4 vertebrae and superior to the T2 vertebrae and/or along the dorsal wall of the esophagus. In some embodiments at least one electrode may be positioned inferior to the C5 vertebrae and superior to the T1 vertebrae. Alternatively or in addition, at least one electrode is positioned 20 centimeters (cm) to 27 cm from the edge of the nasogastric cavity. Further, recruiting at least one sympathetic ganglia may include recruiting one or more of the left stellate ganglion or the right stellate ganglion. In some embodiments, recruiting at least one sympathetic ganglia may including blocking the transmission of nerve signals along the sympathetic ganglia, such as, for example, along the left stellate ganglion or along the right stellate ganglion. In some embodiments, recruitment of the left stellate ganglion may be used in patients presenting with cardiac indications and/or upper left limb pain. In other embodiments, recruitment of the right stellate ganglion may be used in patients with cardiac arrhythmias. The recruitment of both the left stellate ganglion and the right stellate ganglion may be used to treat patients presenting with cardiac arrhythmias such as, for example, ventricular arrhythmia.

The methods may further include monitoring, sensing, and/or testing one or more functions, activity, or other parameters of the brain or nervous system, obtaining the results of the sensing or tests, and analyzing these results, for example, to determine the effect of the transmitted electrical signal on nervous function and/or activity. Based on the test results and their analysis, parameters (e.g., timing, duration, profile, and/or intensity) for the electrical signal may be generated or modified, and one or more nerves may be recruited or released based on the parameters. In some cases, the methods may include testing nervous function and the status of one or more nerve recruitments. Exemplary tests on the status of nerve (e.g., sympathetic ganglia) recruitments include detection of electrodermal activity, monitoring heart rate variability, evaluating responses related to the control of pupil diameter and blood flow to the eye, measuring peripheral blood flow (e.g., via transcranial Doppler ultrasonogram), heart rate and blood pressure variability analysis, thermoregulatory sweat test, magnetic resonance imaging (MRI, e.g., functional MRI), single-photon emission computed tomography (SPECT), evaluation of electroencephalography (EEG) waveforms, the measurement of visual, audio and somatosensory evoked potentials, changes in absolute vital sign values, and changes in pain threshold. The tests may also include detecting chemistry (e.g., levels and activities of proteins or other molecules such as inflammation- or pain—related molecules) in the blood or other bodily fluids. The chemistry tests may include measuring the level and/concentrations of TNF-α, other cytokines, serotonin, gastrin, norepinephrine, and/or other hormones. For example, methods of the present disclosure may include monitoring the recruitment of the sympathetic ganglia by observing palpebral droop, performing an electroencephalogram, measuring pupil diameter, observing a color change in the sclera, measuring skin temperature, performing a transcranial Doppler ultrasonogram, or a combination thereof.

The tests of nerve recruitment may be performed prior to, during, or after an electrical signal is transmitted or at different stages of recruitment signal transmission. For example, the tests may be performed before, during, or after a transmitted electrical signal (e.g., before, during, or after one or more periods of a pulsed electrical signal). Alternatively or in addition, the tests may be performed before, during, or after recruitment of a nerve. Brain function and/or sympathetic ganglia recruitment status or activity may be determined based on the test results. The results from the tests performed at different times may be compared to each other or to reference threshold values or ranges, e.g. thresholds or ranges that indicate normal nervous function or other levels of nervous function. In such cases, brain function and/or sympathetic ganglia recruitment status may be determined based on the comparisons.

For a patient receiving or having received treatment via an electrical signal from an esophageal catheter, the brain function and/or sympathetic ganglia recruitment status in the patient may be detected and compared to parameters indicative of normal function and/or status of the brain and/or the nerves. If a difference or a significant difference is determined, one or more parameters of the electrical signal transmitted by at least one electrode may be modified to adjust the recruitment of the sympathetic ganglia. The adjustment may be performed continuously (e.g., based on real-time monitoring of brain function and/or sympathetic ganglion recruitment status) for delivering optimal and personalized therapy to the patient.

In one or more embodiments, monitoring the recruitment of a nerve (e.g. sympathetic ganglia) may include monitoring the palpebral droop or slump of a patient. When transmission of nerve signals through the sympathetic ganglia is blocked (e.g., when the sympathetic ganglia is recruited) one or more superior eyelids of a patient may droop, slump or distend further from its normal open position. In some instances, blocking the transmission of nerve signals through the left stellate ganglion may cause the left superior eyelid to droop. Additionally or in the alternative, blocking the transmission of nerve signals through the right stellate ganglion may cause the right superior eyelid to droop. Monitoring the palpebral droop of a patient (e.g., observing whether either or both superior eyelids are drooping) may inform a controller and/or a user (e.g., physician or patient) of the recruitment status of one or more nerves, such as, for example, the sympathetic ganglia (e.g., left stellate ganglion and/or right stellate ganglion). As described herein, when a physiological parameter (e.g., palpebral droop) is observed and/or monitored, the observation may be made by one or more sensors and/or a user (e.g., patient, physician, nurse, or other healthcare professional). The one or more sensors and/or users may transmit the observation to a controller, in some embodiments.

In one or more embodiments, monitoring the recruitment of a nerve (e.g. sympathetic ganglia) may include monitoring the brain activity of a patient via an electroencephalogram (EEG). Generally, an EEG records the electrical activity of a brain via electrodes placed along the surface of a scalp or transcutaneously through the scalp. Through the electrodes, an EEG records voltage fluctuations resulting from the current within neurons of the brain. After the transmission of nerve signals through a sympathetic ganglion has been blocked (e.g., when the sympathetic ganglion is recruited) for 5 minutes to 45 minutes (e.g., 5 minutes, 10 minutes, 15 minutes, 20 minutes, 25 minutes, 30 minutes, 40 minutes), decreases in spectral edge frequency (SEF), beta to theta ratio (BTR), median frequency (MF), and beta to delta ratio (BDR) may be detected via EEG. The general operation of an EEG and how to detect decreases in SEF, BTR, MF, and/or BDR is known to those skilled in the art and, for the sake of brevity, will not be described in further detail herein. Monitoring the EEG of a patient (e.g., observing decreases in SEF, BTR, MF, and/or BDR) may inform a controller and/or a user (e.g., physician or patient) of the recruitment status of one or more nerves, such as, for example, the sympathetic ganglia (e.g., left stellate ganglion and/or right stellate ganglion). In some embodiments, a decrease of at least approximately 60%, at least approximately 65%, at least approximately 70%, at least approximately 75%, or at least approximately 80% in SEF, BTR, MF, and/or BDR may indicate recruitment of the sympathetic ganglia. In some embodiments, at least a 10% decrease in the bispectral index (BIS) of a patient, as monitored via EEF, may indicate recruitment of at least one sympathetic ganglion. In other embodiments, at least a 15% decrease or a 10-20% decrease in BIS may indicate recruitment of at least one sympathetic ganglion.

In one or more embodiments, monitoring the recruitment of a nerve (e.g. sympathetic ganglia) may include monitoring the pupil diameter and/or sclera color of a patient. When transmission of nerve signals through the sympathetic ganglia is blocked (e.g., when a sympathetic ganglion is recruited) a pupils of a patient may decrease in diameter. Further, the blocking of the transmission of nerve signals through the sympathetic ganglia (e.g., recruitment of the sympathetic ganglia) may cause one or more sclera of a patient to redden (i.e., change color from white to red or pink). Pupil contraction and sclera reddening may both be the result of increased blood flow to the eye as a result of the sympathetic ganglia recruitment. In some instances, blocking the transmission of nerve signals through the left stellate ganglion may cause the left pupil to contract (e.g., decrease in diameter) and/or the left sclera to redden. Additionally or in the alternative, blocking the transmission of nerve signals through the right stellate ganglion may cause the right pupil to contract (e.g., decrease in diameter) and/or the right sclera to redden. Monitoring the pupil size and/or sclera color of a patient may inform a controller and/or a user (e.g., physician or patient) of the recruitment status of one or more nerves, such as, for example, the sympathetic ganglia (e.g., left stellate ganglion and/or right stellate ganglion). In some embodiments, a difference of greater than or equal to 0.2 mm (e.g., greater than or equal to 0.3 mm or greater than or equal to 0.4 mm) in pupil diameter may indicate recruitment of at least one sympathetic ganglion. In other words, the measurement of one pupil of a patient as having a diameter 0.2 mm greater than the other pupil may indicate sympathetic ganglia recruitment.

In one or more embodiments, monitoring the recruitment of a nerve (e.g. sympathetic ganglia) may include monitoring one or more skin temperatures of a patient. After transmission of nerve signals through the sympathetic ganglia has been blocked (e.g., when a sympathetic ganglion is recruited) for 2 minutes to 30 minutes (e.g., 3 minutes, 5 minutes, 8 minutes, 10 minutes, 13 minutes, 15 minutes, 18 minutes, 20 minutes, 25 minutes, 30 minutes) the skin temperature on a limb and face of a patient may increase by 0.25° C. to 2° C. (e.g., 0.5° ° C. to 1.5° C.). In a healthy patient with normal nervous system function, limb and face temperature is a few degrees Celsius (e.g., 1-5° C.) below core body temperature. When the transmission of nerve signals through a sympathetic ganglion is blocked (e.g., the sympathetic ganglion is recruited), the skin temperature and face temperature of a patient may increase as blood flow increases to the head and limbs. Monitoring the skin temperature of the limbs and/or face of a patient (e.g., observing whether either or both skin temperatures increases) may inform a controller and/or a user (e.g., physician or patient) of the recruitment status of one or more nerves, such as, for example, the sympathetic ganglia (e.g., left stellate ganglion and/or right stellate ganglion).

In one or more embodiments, monitoring the recruitment of a nerve (e.g. sympathetic ganglia) may include monitoring the skin perspiration of a patient. Monitoring the skin perspiration may include monitoring either the rate of sweat or the density of sweat from or on one or more areas of skin. In at least one embodiment, skin perspiration is measured on both limbs of a patient. If either side of the patient has at least a 40% decrease (e.g., at least a 50% decrease or at least a 60% decrease) in skin perspiration rate or skin perspiration density compared to the other side, the sympathetic ganglia may be recruited.

In one or more embodiments, monitoring the recruitment of a nerve (e.g. sympathetic ganglia) may include monitoring blood circulation in the brain of a patient via a transcranial Doppler ultrasonogram. Generally, a transcranial Doppler ultrasonogram measures the velocity of blood flow through a brain's blood vessels by measuring the echoes of ultrasound waves passing from the blood vessels through the cranium. Like other types of Doppler ultrasonography, data or images can be generated based on measuring the Doppler effect of ultrasound waves introduced to the patient, influenced relative to the velocity of a fluid traveling within an anatomical space. After the transmission of nerve signals through a stellate ganglion has been blocked, the velocity of blood circulation in the brain may increase, which can be detected by a transcranial Doppler ultrasonogram. The general operation of a Doppler ultrasonogram is known to those skilled in the art and will not be described in further detail for sake of brevity. Monitoring the transcranial Doppler ultrasonogram of a patient (e.g., observing an increase in blood circulation velocity in the brain) may inform a controller and/or a user (e.g., physician or patient) of the recruitment status of one or more nerves, such as, for example, the sympathetic ganglia (e.g., left stellate ganglion and/or right stellate ganglion). For example, in some embodiments, a decrease in the blood flow in the middle cerebral artery by at least approximately 10% (e.g., at least approximately 15%) may be indicative of recruitment of the sympathetic ganglia. In other embodiments, an increase of at least approximately 15% (e.g., at least approximately 18% or at least approximately 20%) in cerebral perfusion pressure may be evidence of sympathetic ganglia recruitment. Further, a decrease of at least 7.5% (e.g., a decrease of at least 10% or a decrease of at least 15%) in zero flow pressure may indicate recruitment of the sympathetic ganglia.

Any of the previously described examples of monitoring recruitment of a nerve may be used alone or in combination with any other example of monitoring recruitment of a nerve. This monitoring may include monitoring the recruitment of the sympathetic ganglia (e.g., left stellate ganglion and/or right stellate ganglion). Further, in some embodiments, the methods of the present disclosure may include, based on the monitoring the recruitment of the sympathetic ganglia, adjusting the electrical signal from at least one electrode.

In one or more embodiments described herein, a system may include medical devices for performing the methods described in the disclosure. The system, as described below, may include components such as a catheter having a tubular member and one or more electrode assemblies, a signal generator to provide stimulation energy to the electrode assemblies, one or more sensors to sense the condition of the patient, and one or more control components allowing another device (including one or more hardware components and/or software components) or user (e.g., a physician, nurse, other healthcare provider, or a patient) to adjust the parameters of the transmitted electrical signal. The different embodiments of the various system components may be combined and used together in any logical arrangement. Furthermore, individual features or elements of any described embodiment may be combined with or used in connection with the individual features or elements of other embodiments. The various embodiments may further be used in different contexts than those specifically described herein. For example, the disclosed electrode structures may be combined or used in combination with various deployment systems known in the art for various diagnostic and/or therapeutic applications.

The systems and methods described in this disclosure may help to achieve at least one or more of the following to a patient: preventing, modulating, controlling, or treating brain injury. In some embodiments, the systems and methods herein may reduce brain injury via recruitment of the sympathetic ganglia. One or more electrical signals may be used to recruit one or more nerves and interfere with aberrant pain signals and help mitigate cell death. Electrical signals (e.g., pulsed electrical signals) transmitted via electrodes placed proximate to the sympathetic ganglia (e.g., in the esophagus near the left stellate ganglion or the right stellate ganglion) to recruit the sympathetic ganglia and temporarily block afferent signals. The transmitted electrical signal (e.g. the recruitment signal) may have a frequency of 100 hertz (Hz) to 100 kHz, including, for example, a frequency of 1 kHz to 50 kHz or 1 kHz to 30 kHz. Further, the recruitment signal may have an amplitude of 10 micro-amperes (μA) to 20 mA per phase. Other frequencies and amplitudes which may block conduction of axonal nerve signals without causing electrode corrosion or tissue damage are also contemplated.

Based on the monitoring of the recruitment of a sympathetic ganglion, the recruitment signal may be adjusted. For example, in one or more embodiments, a patient may be monitored for one or more indications of a sympathetic ganglion recruitment (e.g., recruitment of the left stellate ganglion and/or recruitment of the right stellate ganglion) as described above, via one or more sensors of the system. In response to a signal from one or more sensors that the sympathetic ganglion is recruited, the recruitment signal may be altered or ceased. For example, the frequency of the recruitment signal might be increased or decreased. Similarly, the amplitude of the recruitment signal might be increased or decreased. In response to a signal that the stellate ganglion is recruited, the recruitment signal might be reduced to a lover level or cease being transmitted. In other embodiments, a recruitment signal might be temporarily stopped if the system receives a signal that the stellate ganglion is recruited. Similarly, in response to a signal from one or more sensors that the sympathetic ganglion is not recruited, the recruitment signal may be altered. For example, the frequency and/or amplitude of the recruitment signal might be increased or decreased.

One or more components of systems described herein may include a plurality of electrodes (e.g., an esophageal catheter comprising a plurality of electrodes). The plurality of electrodes may be similarly shaped or have different shapes. For example, one or more electrodes may be curved, flat, straight-edged, rounded, planar, or other shape. In some embodiments, each of the plurality of electrodes may transmit a signal independently from the other electrodes of the plurality. In other embodiments, two or more electrodes of the plurality may be synced (e.g., configured to transmit coordinated signals). In some embodiments, in response to a signal that one of either the left stellate ganglion or the right stellate ganglion is recruited, the recruitment signal transmitted by one or more electrodes may be adjusted while the recruitment signal transmitted by one or more other electrodes may be not adjusted. For example, in response to a signal that the left stellate ganglion is recruited (when the right ganglion has not yet been recruited), the recruitment signal directed to the left stellate ganglion may be ceased or otherwise modified, while the recruitment signal directed to the right stellate ganglion may not be adjusted. Similarly, in response to a signal the right stellate ganglion is recruited (when the left ganglion has not yet been recruited), the recruitment signal directed to the right stellate ganglion may be ceased or otherwise modified, while the recruitment signal directed to the left stellate ganglion may not be adjusted.

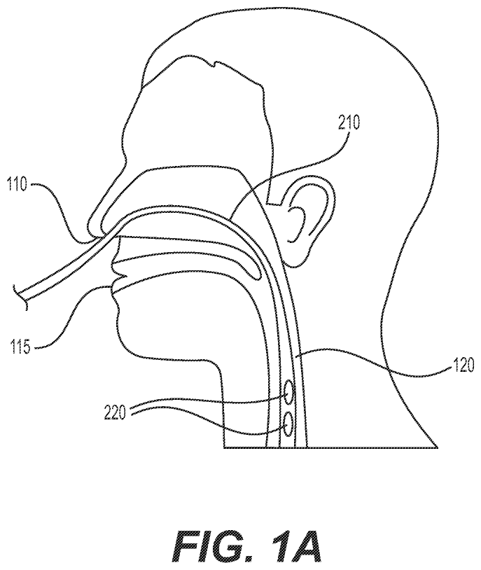

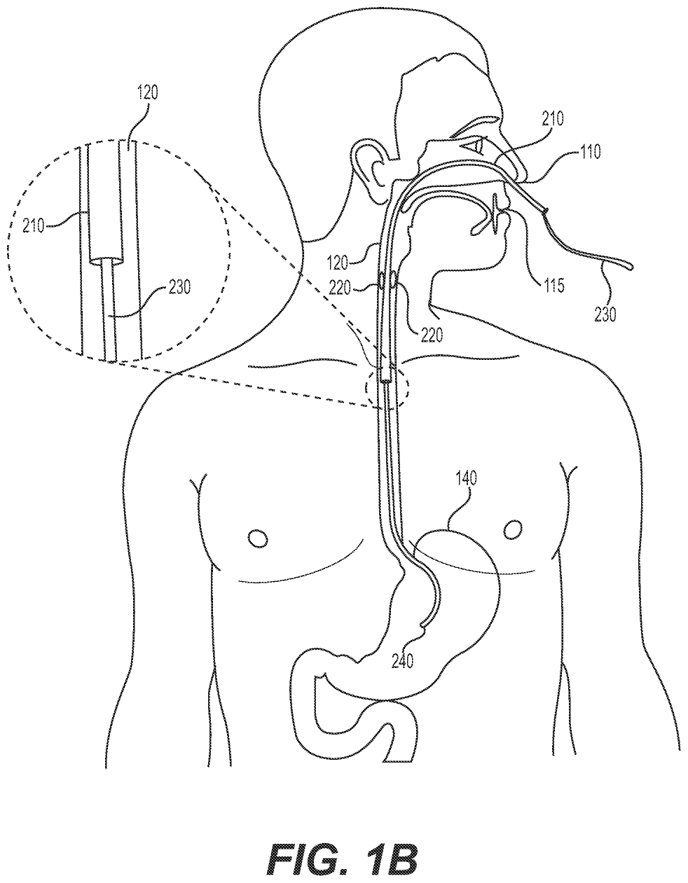

FIG. 1A illustrates the anatomy of the head and neck and, in particular, the relative locations of the nasoesophageal cavity 110, oroesophageal cavity 115, and esophagus 120. In one or more embodiments an esophageal catheter 210, including one or more electrodes 220, may be inserted through a nasoesophgeal cavity 110 and into the esophagus 120. Although not shown in FIG. 1A, in some embodiments, esophageal catheter 210 may be inserted through oroesophageal cavity 115 into esophagus 120. Esophageal catheter 210 may be positioned such that one or more electrodes 220 are proximate to one or more nerves (e.g., sympathetic ganglia).

Referring to FIG. 1B, a system is shown according to one or more embodiments where esophageal catheter 210 is incorporated with or includes a feeding tube 230. According to some embodiments, a feeding tube 230 may travel from the exterior of a patient through the nasoesophgeal cavity 110 or an oroesophageal cavity 115 into the esophagus 120. One or more electrodes 220 of esophageal catheter 210 may be positioned in the esophagus 220 and proximate to one or more nerves (e.g., sympathetic ganglia). A distal end 260 of the feeding tube 230 may extend into the stomach 140 allowing for material to be passed from the exterior of the patient to the stomach 140 via feeding tube 230 without disturbing the placement of the one or more electrodes 220. Esophageal catheter 210 may be placed over an already inserted feeding tube 230, using the feeding tube 230 as a guide for the placement of esophageal catheter 210. In other embodiments, esophageal catheter 210 may be inserted next to and coupled to an already inserted feeding tube 230, e.g., as a side tube. In such embodiments, the esophageal catheter 210 and feeding tube 230 may occupy non-coaxial spaces within an esophagus 120. In some embodiments, electrodes 220 may be positioned, via a biocompatible adhesive or other suitable anchoring mechanism, on an outer surface of feeding tube 230. In further embodiments, esophageal catheter 210 may include a thermal probe to measure one or more internal temperatures.

In one or more embodiments, esophageal catheters 210 are readily applied to, or inserted into, the patient. The placement or insertion may be temporary, and catheter 210 may be easily removed from the patient at a later time without the need for surgery. In some embodiments, esophageal catheter may be left in the esophagus 120 of a patient for greater than or equal to 24 hours, greater than 7 days, greater than 10 days, greater than 14 days, greater than 21 days, greater than 28 days, or even greater than 30 days. For example, esophageal catheter 210 may be withdrawn once the patient is determined to no longer have an SAH and/or once the patient is no longer at risk, or is at a significantly reduced risk, for an SAH-mediated delayed CVS.

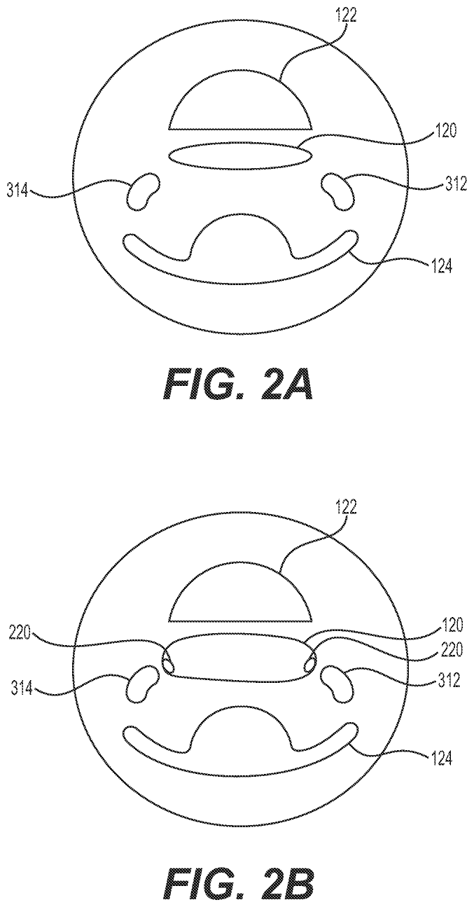

FIG. 2A further illustrates the anatomy of the neck and, in particular, the relative locations of esophagus 120, trachea 122, vertebra 124, left stellate ganglion 314 and right stellate ganglion 312. The cross-section shown in FIG. 2A is representative of any axial cross-section taken in the C4 to T2 region. The sympathetic ganglia includes the left stellate ganglion 314 and the right stellate ganglion 312. Further, each of the left stellate ganglion 314 and the right stellate ganglion 312 may have more than one branch. In some embodiments, vertebra 124 may be the C5 vertebra, the C6 vertebra, or the C7 vertebra.

Referring to FIG. 2B, the same axial cross section of the neck anatomy is taken as FIG. 2A, however FIG. 2B also depicts the placement of electrodes 220 of an esophageal catheter 210. As can be seen in FIG. 2B, esophagus 120 is maintained in an enlarged and distended state while esophageal catheter 210 is inserted therein. One or more electrodes 220 may be positioned proximate to the sympathetic ganglia, including, for example, left stellate ganglion 314 and/or right stellate ganglion 312. In some embodiments, the lumen of esophagus 120 may distend up to approximately 3 cm (e.g., up to approximately 2 cm) in the anterior-posterior dimension and up to approximately 5 cm (e.g., up to approximately 3 cm) in a lateral dimension.

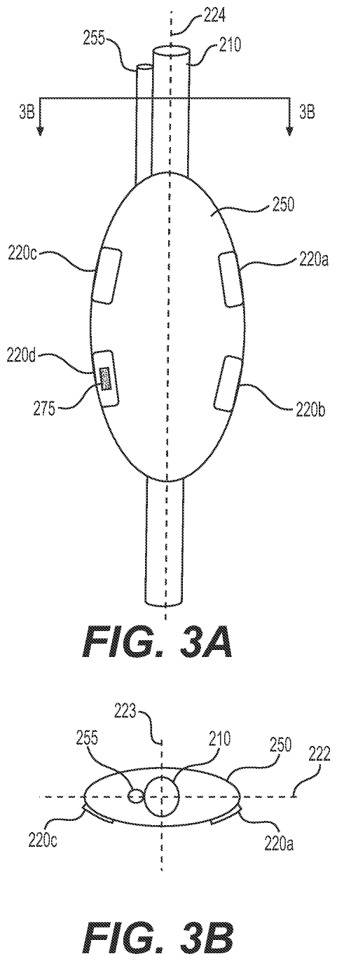

FIG. 3A illustrates an exemplary intermediate section of an esophageal catheter 210 including an inflation lumen 255, inflatable (expandable) member 250, electrodes 220, and feeding tube 230. The inflation member 250 runs along a length of the tubular esophageal catheter (e.g., occupying an intermediate section of the catheter length) and encompasses, encloses, or circumscribes the exterior of feeding tube 230, as shown in cross-section FIG. 3B. Inflation lumen 255 may extend proximally from the inflatable member 250 to the exterior of the patient, where it may be coupled to a controller and/or a fluid providing device or source. Fluid (e.g., air, saline, water) may be transported from the fluid providing device exterior of the patient to the inflatable member 250 via the inflation lumen 255. The transportation of fluid to the inflatable member 250 may cause the intermediate section of esophageal catheter 210 (e.g., the inflatable member 250) to expand from a contracted state to an expanded state. In one example, inflation member 250 may be a compliant or semi-compliant balloon. In other embodiments, an intermediate section may include an actuating member until tensile stress that is operable to expand the intermediate member upon movement and/or adjustment of the actuating member.

Electrodes 220 may comprise traditional electrode metals such as, for example, stainless steel, gold, or an alloy including platinum and iridium. Each electrode may have an exposed surface area greater than or equal to 1.5 mm², such as, for example, greater than or equal to 2 mm², greater than or equal to 2.5 mm², greater than or equal to 3 mm², greater than or equal to 4 mm², or greater than or equal to 5 mm². Electrodes 220 may have a surface area such that a recruitment signal may be transmitted without damage to the electrode 220 or surrounding tissue. Electrodes 220 may be configured to have a surface area such that a transmitted charge density does not exceed therapeutic levels. Electrode 220a is positioned on a surface of the catheter opposite to electrode 220c (on an opposing side of longitudinal axis 224), and both electrodes 220a and 220c may be at the same axial position along longitudinal axis 224. Similarly, electrode 220b is positioned on a surface of the catheter opposite to electrode 220d (on opposing sides of longitudinal axis 224), and both electrode 220b and electrode 220d may be positioned at the same axial position along longitudinal axis 224 (and longitudinally staggered from electrodes 220a and 220c). Electrode 220a may be longitudinally aligned with electrode 220c, and electrode 220b may be longitudinally aligned with electrode 220d. All electrodes 220 may be independently controlled to transmit different electrical signals. In one or more embodiments, electrodes 220a and 220b are configured to transmit the same electrical signal. In some embodiments, two or more electrodes may be configured to be synced, where one electrode serves as the source of the electrical signal transmission and a second electrode may be the sink (e.g., the return electrode). In other embodiments, electrodes 220c and 220d may be configured to transmit the same electrical signal. As seen in FIG. 3B, catheter 210 includes a first lateral axis 222 (a major lateral axis), and a second lateral axis 223 (a minor lateral axis, e.g., an antero-posterior axis). First lateral axis 222 and second lateral axis 223 are both substantially perpendicular to longitudinal axis 224, and are substantially perpendicular to one another. Inflatable member 250 may have a larger dimension in the expanded state along first lateral axis 222 than along second lateral axis 223. Electrodes 220 may be positioned on inflatable member 250 on only one side of first lateral axis 222, while no electrodes 220 are positioned on inflatable member 250 on an opposing side of first lateral axis 250. This positioning may be based on the positions of left stellate ganglion 314 and right stellate ganglion 312 on only one lateral side of esophagus 120 as shown in FIGS. 2A and 2B. Electrodes 220 may be affixed or otherwise coupled to the exterior (outer surface) of esophageal catheter 210. In some embodiments, electrodes 220 may be embedded into esophageal catheter 210 and windows on the catheter may expose electrodes, allowing for a conductive path between electrodes 220 and surrounding tissue, including the esophagus in which catheter is inserted. Alternatively, electrodes could be printed onto the surface of the catheter by one of several known means (e.g. conductive inks, polymers) as described in U.S. Pat. No. 9,242,088, which is incorporated by reference herein. Further, the electrodes may be integrated into a flexible printed circuit, which can be attached to, or integrated into, the catheter. Insulation means known in the art may be used to ensure that the electrodes, and not any unwanted electrical elements, are exposed to direct contact with the patient. Esophageal catheter 210 may include rows of apertures or windows positioned proximally, medially and distally, such that when esophageal catheter 210 is inserted into an esophagus, at least one window may face, abut, or be positioned in the vicinity of the left stellate ganglion 314, at least one window may face, abut, or be positioned in the vicinity of the right stellate ganglion 312, and/or at least one window may face, abut, or be positioned in the vicinity of the sympathetic ganglia. Electrodes 220 may extend partially around the circumference of esophageal catheter 210. This electrode configuration may allow electrodes 220 to target a desired nerve for stimulation while minimizing application of electrical charge to undesired areas of the patient's anatomy (e.g., other nerves or the heart). The oral catheter may include one or more features (anchor, hooks, biocompatible adhesive, or the like) to secure or stabilize the catheter within the patient, and or the electrodes 220 at a specific location. Further, esophageal catheter 210 may be shaped to conform to the inner wall of the esophagus and position at least one electrode proximate the sympathetic ganglia. In some embodiments, esophageal catheter 210 is shaped to dispose at least two electrodes proximate to each of the left stellate ganglion and right stellate ganglion.

The dimensions of esophageal catheter 210 may be customized in accordance with the anatomy of a particular patient (e.g., different sizes of humans, pigs, chimpanzees, etc.). The catheter may have a length greater than or equal to 20 cm, such as for example, 25 cm, 30 cm, 35 cm, 40 cm, 45 cm, or 50 cm. Esophageal catheter 210 may also have an outer diameter less than or equal to 10 mm, such as for example, less than or equal to 8 mm, less than or equal to 6 mm, less than or equal to 5 mm, or less than or equal to 4 mm.

Esophageal catheter 210 may incorporate markings or other indicators on its exterior to help guide the positioning and orientation of the device. The catheter may also include internal indicators (e.g., radiopaque markers, contrast material such as barium sulfate, echogenic markers) visible by x-ray, ultrasound, or other imaging technique to assist with positioning esophageal catheter 210 in the desired location. In one or more embodiments, a radiopaque marker 275 may be placed on or adjacent to an electrode 220, as shown in FIG. 3A. In some embodiments, markers may correspond to positions of electrodes on only one side of lateral axis 222. Esophageal catheter 210 may include any combination of the features described herein. Accordingly, the features of the catheter are not limited to any one specific combination shown in the accompanying drawings.

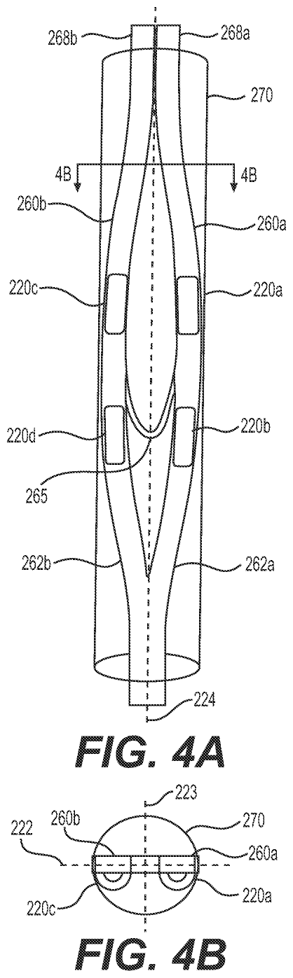

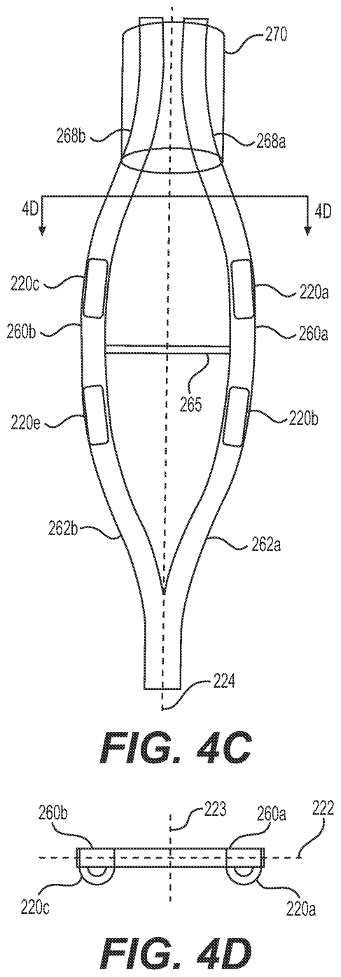

As previously stated, aspects of one embodiment may be combined with aspects of one or more other embodiments. For example, the description of electrodes 220, their integration into esophageal catheter 210, and their placement proximate to sympathetic ganglia (e.g., left stellate ganglion 314 and/or right stellate ganglion 312) may be applicable to any esophageal catheter described herein. Referring now to FIGS. 4A-4B, an esophageal catheter 410 may include an intermediate section comprising two or more expandable arms 260 connected by a tether 265, which is slack in the contracted state shown in FIGS. 4A and 4B. In a contracted state, at least the intermediate section of catheter 410 is disposed within a sheath 270. Each arm 260 may include a joined end 262, a proximal end 268, and at least one electrode 220 positioned between the joined end 262 and the proximal end 268. Each joined end 262 may connect to the joined ends 262 of the other arms 260 at the distal end of catheter 410. For example, joined end 262a of arm 260a connects to joined end 262b of arm 262b by a weld or other suitable connection, as shown in FIG. 4A. Further, the proximal end 268 of each arm 260 is further displaced from the proximal end 268 of the other arms 260 when in an expanded state as compared to in a contracted state. For example, proximal end 268a of arm 260a is farther displaced from proximal end 268b of arm 260b when the intermediate section of esophageal catheter 210 expands to an expanded state, as shown in FIGS. 4A-4B.

Another exemplary intermediate section of esophageal catheter 210 shown in FIGS. 4A-4B is in a contracted state and disposed in a sheath 170. The shape of the catheter may be formed by heat setting the sheath 170, or by adding a shaped stainless steel wire or a shape memory nitinol wire or any other shape memory alloy. A shape-memory alloy may activate a helical shape of esophageal catheter 210 when heated to a temperature of 30° C. to 45° C., such as, for example, 37° ° C. When esophageal catheter 210 is deployed and removed from sheath 170 the proximal ends 268 of the intermediate section of the catheter may expand and displace farther apart, as shown in FIGS. 4C-4D. In other words, catheter 210 may be biased into the expanded state. As shown in FIG. 4C, the tether 265 is stretched taut, limiting the extent to which proximal ends 268 may radially separate and expand away from one another. This expanded helical shape may help anchor esophageal catheter 210 to the esophagus and/or to stabilize the catheter during nerve recruitment. The helical shape may position electrodes 220 at different radial positions within the esophagus and relative to target nerves such as for example the left stellate ganglion and/or right stellate ganglion. In some embodiments, all electrodes of an esophageal catheter 210 may be positioned on one side of the first lateral axis 222.

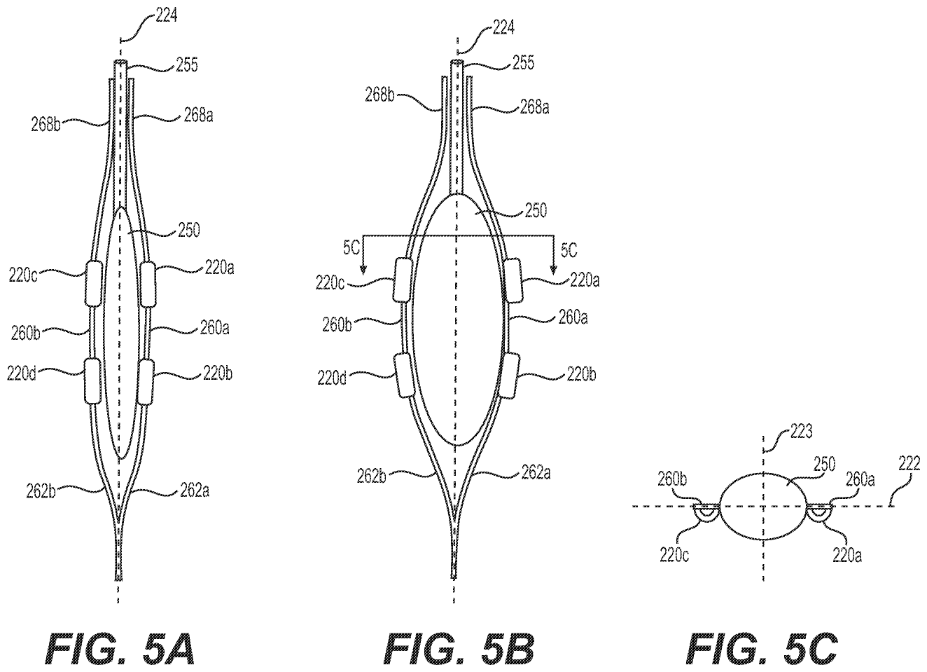

Referring now to FIG. 5A, an exemplary intermediate section of an esophageal catheter 210 in a contracted state is shown. FIGS. 5B-5C show the same intermediate section of esophageal catheter 210 in an expanded state. The catheter may include an intermediate section comprising two or more arms 260 on opposing sides of inflatable member 250 and an inflation lumen 255 connected to the inflatable member 250. Each arm 260 may include a joined end 262, a proximal end 268, and at least one electrode 220 positioned between the joined end 262 and the proximal end 268. Each joined end 262 may connect to the joined ends 262 of the other arms 260. For example, joined end 262a of arm 260a connects to joined end 262b of arm 262b, as shown in FIG. 5A. Further, the proximal end 268 of each arm 260 is further displaced from the proximal end 268 of the other arms 260 when in an expanded state as compared to in a contracted state. For example, proximal end 268a of arm 260a is farther displaced from proximal end 268b of arm 260b when the intermediate section of esophageal catheter 210 expands to an expanded state, as shown in FIGS. 5A-5B.

Inflatable member 250 may be inflated in a substantially similar manner as described above with respect to FIGS. 3A and 3B. The transportation of fluid to the inflatable member 250 may cause the intermediate section of esophageal catheter 210 (e.g., the inflatable member 250 and arms 260) to expand from a contracted state to an expanded state. In some embodiments, the electrodes 220 may be on only one side of lateral axis 222.

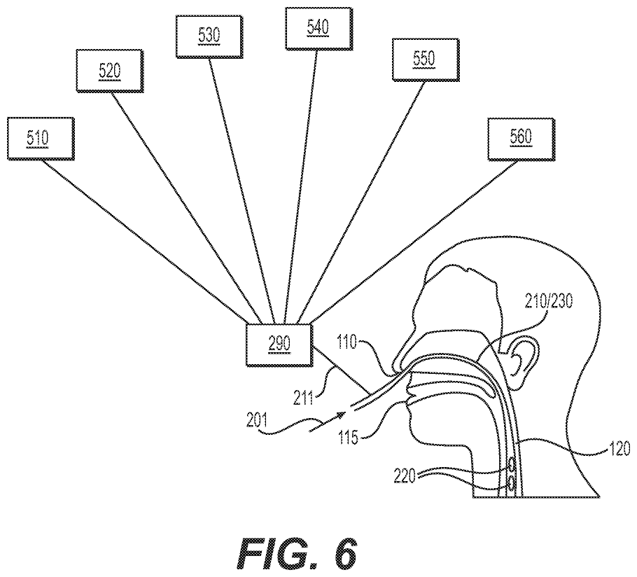

FIG. 6 illustrates a block diagram of the various components of system. The system may include a controller 290, which may be part of any of the control units described herein. Each of the components of the system may be operably coupled to controller 290, and controller 290 may manage operation of electrodes 220 during nerve recruitment and control the gathering of information by various sensors (510, 520, 530, 540, 550, 560) during monitoring of recruitment. It should be understood that the various modules described herein may be part of a computing system and are separated in FIG. 6 for explanatory purposes only; it is not necessary for the modules to be physically separate.

Controller 290 may connect to the esophageal catheter 210 via a lead wire cable 211 and a connector. The lead wire cable may have a length greater than or equal to approximately a meter and the esophageal catheter may optionally include a feeding tube 230, with an inlet 201 for administration of substances including food stuff and nutrients. Controller 290 may connect to one or more sensors. For example, a sensor which detects palpebral droop 510, electrodes of an EEG 520, sensors which measure pupil diameter 530 (including, e.g., a pupilometer), a sensor which detects sclera color change 540, one or more skin temperature sensors 550, or sensors connected to a transcranial Doppler ultrasonogram 560, as described herein. In some embodiments, one or more sensors configured to detect a physiological parameter (e.g., palpebral slump, pupil diameter, sclera color) may include a camera or other imaging device, one or more photo filers, image recognition/processing software, and/or an algorithm for determining the state of a physiological parameter based on a captured image. For example, an infrared camera may be used to monitor skin temperatures over time and one or more image recognition software components or algorithms may recognize or detect one or more patterns in the fluctuation of skin temperature. In further embodiments, one or more sensors for detecting skin temperature may include one or more triple-patch electrodes placed on the forehead and/or one or more limbs. In some embodiments, an electrode, functioning as a sensor, may measure intraocular pressure. In still other embodiments, one or more electrodes or collection vesicles may be placed near or on the skin to measure skin perspiration rate and/or perspiration density.

The one or more sensors may transmit various signals to the operator 290, influencing the operator 290 to adjust the electrical signal transmitted by one or more electrodes 220. For example one or more sensors may transmit a signal to the operator 290 and based on that signal, the operator 290 may induce one or more electrodes to adjust the amplitude or frequency of the transmitted electrical signal.

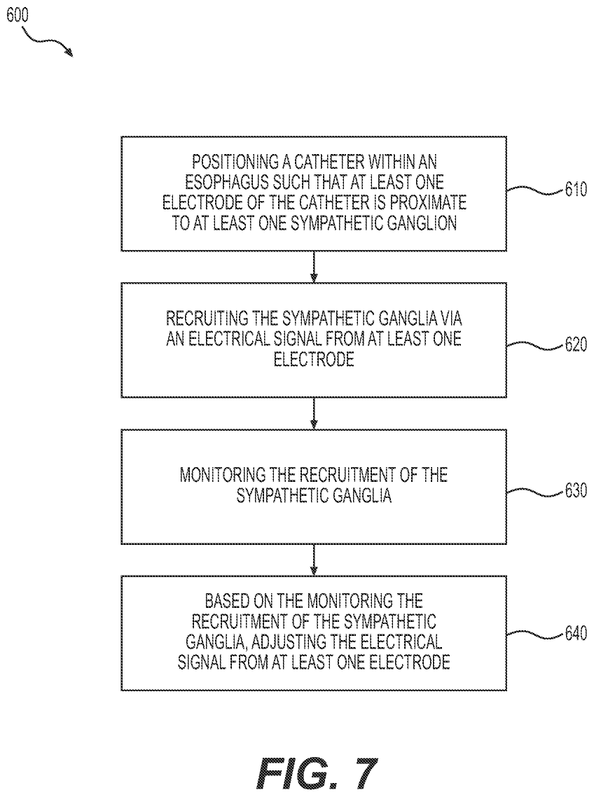

FIG. 7 depicts a flow chart of an exemplary method 600 of treatment, according to the present disclosure. In the discussion below of exemplary methods and flow charts, reference may be made to the reference numerals detailed in FIGS. 1A-6. In one or more embodiments, a method of treatment may include positioning a catheter 210 within an esophagus 120 such that at least one electrode 220 of the catheter 210 is proximate to at least one sympathetic ganglion (e.g., left stellate ganglion 314 and/or right stellate ganglion 312) (step 610). The method 600 may further include recruiting the sympathetic ganglia (e.g., left stellate ganglion 314 and/or right stellate ganglion 312) via an electrical signal from at least one electrode (step 620). In some embodiments, the method 600 may further include monitoring the recruitment of the sympathetic ganglia (e.g., left stellate ganglion 314 and/or right stellate ganglion 312) (step 630). This monitoring may optionally be done by one or more of a sensor which detects palpebral droop 510, electrodes of an EEG 520, sensors which measure pupil diameter 530, a sensor which detects sclera color change 540, one or more skin temperature sensors 550, or sensors connected to a transcranial Doppler ultrasonogram 560. The method 600 may further include, based on the monitoring the recruitment of the sympathetic ganglia (e.g., left stellate ganglion 314 and/or right stellate ganglion 312), adjusting the electrical signal from at least one electrode 220 (step 640).

In some embodiments, recruitment of the sympathetic ganglia via high-frequency electrical signals using temporary intra-esophageal electrodes may provide for a simpler, more convenient, and safer method to transiently and reversibly block the transmission of sympathetic nerve signals than the current standard of care (e.g., percutaneous hypodermic needle injection of anesthesia.

One or more esophageal catheters described herein may have features to meet unique anatomical and electrical requirements. Intra-esophageal lumens, such as, for example, nasogastric feeding tubes or diaphragmatic EMG sensing leads used for neurally adjusted ventilator assist may be inserted in patients and safely left in the patient for days and may be removed without harm to the patient. Esophageal catheters described herein may minimize the conduction distance from the stimulation source electrodes to the target sympathetic neurons, maximizing the field strength directed in a lateral and dorsal direction towards target tissue. This may minimize unwanted stimulation of other neural structures, such as, for example, the recurrent laryngeal nerves which are located more medially and anteriorly, and may minimize the risk of causing mechanical or electrical damage to the esophageal wall or neural structures.

The amplitude of the electrical signal transmitted by one or more electrodes of an esophageal catheter (e.g., a recruitment signal) may be graded to select which fibers in a nerve are recruited, based on fiber size and fiber location. Recruitment of a nerve may be suddenly onset (e.g., may occur in less than or equal to 10 ms, such as, less than or equal to 5 ms), may be rapidly released, and/or may persist minutes or hours after recruitment signal is ceased. For example, in some embodiments, a recruitment signal lasting one minute in duration may recruit at least one sympathetic ganglion for at least approximately 30 minutes.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

1-24. (canceled)

25. A system comprising:
an esophageal catheter including at least one electrode;
a sensor; and
a controller in communication with the at least one electrode and the sensor;
wherein, upon receiving a signal from the sensor, the controller induces the electrode to transmit a recruiting signal that recruits a cervical ganglion.

26. The system of claim 25, wherein the sensor includes one or more electroencephalogram electrodes.

27. The system of claim 26, wherein the signal received by the controller, from the sensor, includes a first EEG measurement comprising a first spectral edge frequency, a first beta to theta ratio, a first median frequency, a first beta to delta ratio, or a combination thereof.

28. The system of claim 27, wherein the controller is configured to compare the first EEG measurement to a second EEG measurement, a reference value, or a reference range.

29. The system of claim 28, wherein the recruiting signal includes a frequency and an amplitude, and the controller is configured to adjust the frequency and/or the amplitude of the recruiting signal, based on the comparison.

30. The system of claim 25, wherein the sensor is configured to measure a blood circulation velocity in a blood vessel in a brain.

31. The system of claim 25, wherein recruiting the cervical ganglion includes blocking transmission of nerve signals along the cervical ganglion.

32. A system comprising:
an esophageal catheter including an electrode;
a sensor; and
a controller in communication with the electrode and the sensor;
wherein upon receiving a first signal from the sensor, or a user, the controller induces the electrode to transmit a recruiting signal to a cervical ganglion, where the recruiting signal is pulsed, and has a frequency of 100 Hz to 100 kHz and an amplitude of 10 μA to 20 mA; and
upon receiving a second signal from the sensor, or the user, the controller induces the electrode to adjust the frequency of the recruiting signal, the amplitude of the recruiting signal, or both.

33. The system of claim 32, wherein the electrode is a first electrode positioned on a surface of the catheter, and the catheter further comprises a second electrode opposite the first electrode, where the first electrode and second electrode are positioned at a same axial level of the catheter.

34. The system of claim 33, wherein the first electrode is on a first side of a lateral axis bisecting the catheter, and the second electrode is on a second side of the lateral axis bisecting the catheter.

35. The system of claim 33, wherein the catheter includes an intermediate section operable to radially expand from a contracted state to an expanded state, and wherein the first electrode and the second electrode are disposed within the intermediate section of the catheter.

36. The system of claim 35, wherein, when the intermediate section of the catheter is in the expanded state, the first electrode is farther displaced from the second electrode, as compared to when the intermediate section of the catheter is in the contracted state.

37. The system of claim 32, wherein the catheter further includes a feeding tube.

38. The system of claim 32, further comprising positioning the catheter such that the electrode is within an oroesophageal cavity.

39. A system comprising:
an esophageal catheter including an electrode;
a sensor for measuring a pupil diameter, a blood circulation velocity in a blood vessel in a brain, a skin temperature, a perspiration rate, or a perspiration density; and
a controller in communication with the electrode and the sensor;
wherein, upon receiving a first signal from the sensor, or a user, the controller induces the electrode to transmit a recruiting signal that recruits a cervical ganglion.

40. The system of claim 39, wherein the sensor includes an infrared monitoring device.

41. The system of claim 39, wherein the controller is configured to compare a first pupil diameter measured by the sensor to a second pupil diameter measured by the sensor, and based on the comparison, adjust an amplitude or a frequency of the recruiting signal.

42. The system of claim 39, wherein the controller is configured to compare a first blood circulation velocity measured by the sensor to a second blood circulation velocity measured by the sensor, and based on the comparison, adjust an amplitude or a frequency of the recruiting signal.

43. The system of claim 39, wherein the controller is configured to:
compare a first skin temperature measured by the sensor to a second skin temperature measured by the sensor;
compare a first perspiration rate measured by the sensor to a second perspiration rate measured by the sensor; or
compare a first perspiration density measured by the sensor to a second perspiration density measured by the sensor; and
based on the comparison, adjust an amplitude or a frequency of the recruiting signal.

44. The system of claim 39, wherein the controller is configured to compare a pupil diameter, a blood circulation velocity, a skin temperature, a perspiration rate, or a perspiration density measured by the sensor to a threshold value or a range, and based on the comparison, adjust an amplitude or a frequency of the recruiting signal.
